(19) United States
(12) Patent Application Publication
(10) Pub. No.: US 2007/0148166 A1
(43) Pub. Date: Jun. 28, 2007

(54) SCFVS IN PHOTOSYNTHETIC MICROBES

(76) Inventors: Madeline Chang Sun Wu, Hong Kong (HK); Sally Ka Kan Mak, Hong Kong (HK); Ken Wan Keung Lau, Hong Kong (HK); Jianping Ren, Hong Kong (HK)

Correspondence Address: BAKER & MCKENZIE LLP, Pennzoil Place, South Tower, 711 Louisiana, Suite 3400, HOUSTON, TX, 77002-2716, US

(21) Appl. No.: 11/548,698

(22) Filed: Oct. 11, 2006

Related U.S. Application Data
(63) Continuation-in-part of application No. 10/144,557, filed on May 13, 2002, now abandoned.

(51) Int. Cl. A61K 39/395; A61K 36/02; C12P 21/06; C12N 1/12

(52) U.S. Cl. 424/133.1; 435/69.1; 435/257.2; 424/195.17

(57) ABSTRACT

Expression of functionally active recombinant single chain antibodies in prokaryotic and eukaryotic heterotrophic algae for pathogen treatment or control in aquaculture and agriculture applications is disclosed; and subsequent modification of the strains to generate transgenic algae propagated under defined conditions.

### CROSS-REFERENCE TO RELATED APPLICATIONS

This is a continuation-in-part of application Ser. No. 10/144,557, filed on May 13, 2002, incorporated herein by reference.

### STATEMENT REGARDING FEDERALLY SPONSORED RESEARCH

Not applicable.

### REFERENCE TO A COMPACT DISK APPENDIX

Not applicable.

### FIELD OF THE INVENTION

The invention relates to the generation of recombinant genes coding for Single chain Fragment variable antibodies (ScFvs) that bind specifically to aquaculture pathogens and the expression of recombinant ScFvs genes in prokaryotic and eukaryotic photosynthetic microbes.

### BACKGROUND OF THE INVENTION

Aquaculture is a rapidly expanding industry. The demand for aquaculture products is rising continuously because the wild population for many cultivated species are declining. Aquaculture species, such as prawn, abalone or the like, are susceptible to bacterial, viral and parasitic diseases. In dense culture situations used for aquaculture, infections often transmit rapidly, inflict massive mortality, and cause large economic losses. If there is a disease outbreak, the pathogen may persist for a long time despite attempts to remove them by expensive pond drying, sun exposure, and installation of water treatment systems. Aquaculture pathogens are not only dangerous to the aquaculture species, some pathogens, such as Vibrio cholerae, can also cause diseases in humans. Antibiotics and/or chemical treatments have been used to control pathogens, but these are not effective for all types of pathogens. Chemical treatments cannot differentiate pathogens from benign species and can be detrimental to the aquaculture species. The removal of benign probiotic species often makes the aquaculture species more susceptible to subsequent infections. Pathogens, such as Vibrio spp., have developed resistance to antibiotics further detracting from such treatments. To prevent the spread of antibiotic-resistant pathogens, many countries have banned the import of farmed species exposed to antibiotics or chemicals and highly sensitive detection methods for both were developed. Additionally, chemical compounds can accumulate in aquaculture ponds and the environment exemplifying the need to limit use of these compounds. Alternative methods to control pathogens in aquaculture are needed.

One alternative is immunization of aquaculture species. Immunization of livestock and poultry has become standard for traditional farm animals. Immunization of animals with a specific antigen to induce immune response and disease control is a known process in humans, cattle, chickens and other animals. Unfortunately, the immune response of aquaculture species are not well documented, and field data clearly show immunization is inadequate for aquaculture conditions. Immune response, even in farm animals, is not rapid enough to prevent disease outbreak, especially with high density populations. The logistics of immunization in aquaculture species are also prohibitive. Thus, traditional immunization in aquaculture species is not practical.

In theory, monoclonal antibodies could be produced in large quantities using hybridomas and traditional antibody production techniques, i.e. mouse, rabbit or goat antibodies. Although theoretically plausible, this method would not be practical. Production using traditional techniques could not be economically increased to the scale required for aquaculture pathogen treatment.

An alternative to traditional antibody production is the use of Single chain Fragment variable antibodies (ScFvs). ScFvs antibodies retain full antigen-binding activity and a recombinant cDNA coding for specific ScFvs can be prepared using standard techniques. The single chain antibodies consist of variable light chain domain and heavy chain domains of an antibody molecule fused by a flexible peptide linker as described in U.S. Pat. No. 5,863,765, the teachings of which are incorporated herein by reference. Many single chain antibodies are effective for controlling human diseases; over 100 therapeutic antibodies are currently in clinical trials for cancer, viral, autoimmune, and other diseases as described by Boder et al., 2000, the teachings of which are incorporated herein by reference.

White Spot Syndrome Virus (WSSV) is an extremely virulent disease that causes considerable economic loss for the shrimp aquaculture industry (Lightner, 1996). WSSV has also been named as white spot baculovirus (WSBV). Other viruses, such as systemic ectodermal and mesodermal baculovirus (SEMBV) (Wongteerasupaya, et al. 1995), penaeid rod-shaped DNA virus (PRDV) (Inouye et al., 1996) and hypodermal and hematopoietic necrosis baculovirus (HHNBV) (Huang et al., 1995), are very closely related to WSSV.

WSSV infection was first reported in Taiwan in 1992 (Chen 1995), the virus spread rapidly to aquacultures in Asia and Indo-Pacific. The WSSV spread rapidly and was first detected in shrimp farms in Texas in 1995, and in January 1999, the virus was detected in samples collected from Nicaragua, Guatemala, Honduras, and the American Pacific coast (Aguirre et al., 2000). By the end of the century, WSSV was globally disseminated. Thus the WSSV is extremely virulent and spreads rapidly.

Shrimp infected with WSSV suffer lethargy, accumulate in shallow water, and swirl. After white spots are first identified on the exoskeleton of an infected shrimp, mass mortality could destroy up to 100% of the culture within 2 to 7 days (Chou et al., 1995). WSSV has a broad host range that includes fresh water or marine shrimp, crabs, crayfish and other arthropods. Penaeid shrimp are particularly vulnerable. After infection, WSSV circulates the hemolymph and infects stomach, integument, abdominal muscles, pleopod, periopod and tissues of the heart, gills, eyestalk, midgut, hepatopancrease, lymphoid organs, nerve cord, spermatophore, testes and ovary. Thus WSSV is extremely lethal and damages or destroys all systems of the infected aquaculture.

WSSV belongs to the genus Whiposvirus within the family Nimaviridae, referring to the thread-like extension on the virus particle. The universal database of International Committee of Taxonomy of Viruses (ICTV) at www.ictvdbiacr.ac.uk/index.htm contains information on Nimaviridae and other viruses (incorporated herein by reference). WSSV virions show an average size of 320×120 nm and contain a rod-shaped nucleocapsid of about 350×80 nm. The nucleocapsids contain a DNA-protein core bounded by a distinctive capsid layer. Among the WSSV isolates collected worldwide, few genetic variations were detected (Lo et al., 1999). One example used herein, isolated from Xiamen, China, contains a 305 kb double-stranded circular DNA encoding approximately 181 open reading frames (ORFs) (Yang et al., 2001). Thus markers and coat proteins for the WSSV are conserved and provide stable targets for antibody production.

Aquaculture pathogens can be extremely lethal, spread rapidly, and are damaging to aquacultures worldwide. A method of controlling and treating aquaculture pathogens effectively without costly chemicals or expensive equipment is required. The present invention provides heterotrophic algae that express recombinant single-chain antibodies for the treatment of aquaculture diseases.

### BRIEF SUMMARY OF THE INVENTION

The present invention provides prokaryotic and eukaryotic algae that produce single-chain antibodies for the treatment of aquaculture pathogens. Additionally, heterotrophic algae may be used to produce large quantities of single-chain antibodies for other treatments. The heterotrophic algae and single-chain antibodies produced may be used to remove or inhibit aquaculture pathogens.

In one embodiment, recombinant Synechocystis or Chlamydomonas express ScFvs with a high specificity for progesterone. In another embodiment Synechocystis PCC 6803 expresses recombinant ScFvs that bind WSSV proteins. Further, ScFvs produced in Synechocystis are used to neutralize WSSV in aquaculture.

One embodiment of the present invention describes heterotrophic algae that express an active ScFv.

A method of inhibiting an aquaculture pathogen is described where generating a heterotrophic algae comprising a recombinant DNA encoding a single chain fragment variable (ScFv) antibody operably linked to a promoter, wherein said heterotrophic algae produces an active ScFv antibody, inoculating an aquaculture with the heterotrophic algae (a), and expressing said ScFv antibody thereby inhibiting said aquaculture pathogen.

A recombinant single chain fragment variable (ScFv) antibody that binds specifically to an aquaculture pathogen is produced by generating heterotrophic algae comprising a recombinant DNA encoding a single chain fragment variable (ScFv) antibody operably linked to a promoter, wherein said heterotrophic algae produces an active ScFv antibody, inoculating an aquaculture with the heterotrophic algae, and expressing said ScFv antibody thereby inhibiting said aquaculture pathogen.

Heterotrophic algae are selected from the group consisting of Chlamydomonas, Synechococcus, Thermosynechococcus, Synechocystis, Euglena, Gloeobacter, Nostoc, Anabaena, Trichodesmium, Prochlorococcus, Chlorella, and Pseudendoclonium.

ScFv antibodies can be generated that specifically bind to progesterone, VP26, or VP28, White Spot Syndrome Virus (WSSV), white spot baculovirus (WSBV), cholera, systemic ectodermal and mesodermal baculovirus (SEMBV), penaeid rod-shaped DNA virus (PRDV), hypodermal and hematopoietic necrosis baculovirus (HHNBV), or other viruses.

Integration cassettes encode (A) a promoter, (B) an ScFv antibody coding region, and (C) a stop codon. The integration cassette may be flanked by host DNA of about 400 nucleotides in length to facilitate incorporation of the ScFv DNA sequence into the host genome.

ScFv antibodies can be ScFv 35, ScFv 85, ScFv 86, DB3, or other ScFv antibody optimized for expression in a heterotrophic algae.

In one embodiment Synechocystis incorporate an integration cassette comprising a Synechocystis psbA II promoter, an ScFv antibody coding region, and a psbA II stop codon wherein said integration cassette allows specific chromosomal integration of the recombinant DNA.

In another embodiment Chlamydomonas, incorporate a site-specific integration cassette with, a promoter region of RbcS2 promoter, the 5′untranslated leader of tobacco mosaic virus, the first intron of RbcS2, an ScFv antibody coding region and the ER retention sequence KDEL (SEQ ID NO: 14), wherein said integration cassette allows specific chromosomal integration of the recombinant DNA.

The above summary of the present invention is not intended to represent each embodiment or every aspect of the present invention.

### BRIEF DESCRIPTION OF THE DRAWINGS

A more complete understanding of the method and apparatus of the present invention may be obtained by reference to the following “Detailed Description” when taken in conjunction with the accompanying “Drawings” herein:

FIG. 1: Amplification of VP26 coding sequence.

FIG. 2: SDS-PAGE of VP26 and VP28. (A) VP 26 and VP 28 in crude protein extract and (B) purification of r-VP26 and VP28.

FIG. 3: Assembly of VP26 ScFv from VL and VH.

FIG. 4: Recombinant expression cassette for Synechocystis 6803.

FIG. 5: Southern blot of rjbJ gene.

FIG. 6: Western blot of ScFv-DB3 in Synechocystis 6803 ScFv-DB3.

FIG. 7: Specificity of ScFv-DB3 expressed in Synechocystis 6803 ScFv-DB3.

FIG. 8: Progesterone conjugate affinities of ScFv-DB3.

FIG. 9: Structure of the progesterone conjugates.

FIG. 10: Recombinant expression cassette for C. reinhardtii 950.

FIG. 11: In vivo neutralization of WSSV using ScFvs.

### DESCRIPTION OF THE PREFERRED EMBODIMENTS

“Single chain Fragment variable antibodies” or “ScFvs” as used herein refer to variable light chain domain and heavy chain domains of an antibody fused by a flexible peptide linker as described in U.S. Pat. No. 5,863,765, the teachings of which are incorporated herein by reference. ScFvs retain full antigen-binding activity and are encoded by a single recombinant DNA molecule. Although ScFvs retain full antigen-binding activity, they lack the complex assembly requirements and are suitable for expression in a variety of organisms. ScFvs for controlling human diseases are described by Boder, et al., (2000), the teachings of which are incorporated herein by reference. Of the over 1400 ScFvs listed in the NCBI™ database, several are listed in TABLE 1.

TABLE 1
SINGLE CHAIN ANTIBODIES

| Species | Single Chain Antibody | Acc # |
|---|---|---|
| Mouse | Catalytic antibody | AAA69766 |
| Human | Collagenase IV binding | BAA19453 |
| Artificial | Streptavidin fusion | CAA77104 |
| Artificial | Anti-rice stripe virus protein | AAG28706 |
| Artificial | BU1 Immunoprophylaxis | AAG39978 |
| DB3 | Progesterone binding ScFv | He, et al., 1995 |

“Neutralization” or “viral neutralization” is the process by which an antibody neutralizes the infectivity of a virus. The antibody may coat the virus forming a stable complex or may cause conformational changes in viral structural proteins on binding; either process may interfere with binding of the virion to cellular receptor sites and entry into the cell.

The terms “complementary” and “complement”, as used herein refer to polynucleotide sequences that are capable of base pairing with contiguous polynucleotide sequences due to sequence homology throughout the complementary regions. Various lengths of DNA will hybridize based on % homology, GC content, and annealing conditions. Hybridization may be observed with greater than 80%, 85%, 90%, 95%, or 99% homology. Sequences with 100% homology are an exact complement. Homology is inversely related to % identity below.

The term “PCR”, as used herein, refers to the polymerase chain reaction. PCR is a method of amplifying a DNA sequence using a heat stable polymerase and a pair of primers, one primer complementary to the (+) strand at one end of sequence to be amplified and the other primer complementary to the (−) strand at the other end of sequence to be amplified. Newly synthesized DNA strands can subsequently serve as templates for the same primer sequences and successive rounds of primer annealing, strand elongation, and dissociation produce rapid and highly specific amplification of the desired sequence.

The term “primer”, as used herein, refers to a short single-stranded oligonucleotide capable of hybridizing to a complementary sequence in a DNA sample. The primer serves as an initiation point for template dependent DNA synthesis. A DNA polymerase can add deoxyribonucleotides to a primer. A “primer pair” or “primer set” refers to a set of primers including a 5′ upstream primer that hybridizes with the 5′ end of the DNA sequence to be amplified and a 3′ downstream primer that hybridizes with the complement of the 3′ end of the DNA sequence to be amplified.

As used herein, the terms “photosynthetic microbes” or “heterotrophic algae” refer to both prokaryotic and eukaryotic strains that can carry out photosynthesis. Some examples of heterotrophic algae are listed in TABLE 2.

TABLE 2
HETEROTROPHIC ALGAE

| Order | Species | Acc # |
|---|---|---|
| Chlorophyta | Chlamydomonas reinhardtii | NC_005353 |
| Chroococcales | Synechococcus sp. | NC_005070 |
| Chroococcales | Thermosynechococcus elongatus | NC_004113 |
| Cyanobacteria | Synechocystis sp. | NC_004967 |
| Euglenida | Euglena longa | NC_002652 |
| Gloeobacteria | Gloeobacter violaceus | NC_005125 |
| Nostocales | Nostoc sp. | NC_003272 |
| Nostocales | Anabaena variabilis | NC_007413 |
| Oscillatoriales | Trichodesmium erythraeum | NC_008312 |
| Prochlorales | Prochlorococcus marinus | NC_007577 |
| Trebouxiophyceae | Chlorella vulgaris | NC_001865 |
| Ulvophyceae | Pseudendoclonium akinetum | NC_008114 |

As used herein, the term “mutant strain” refers to a non-wild type strain. In certain embodiments of the invention, a mutant strain produces a new gene product that is not native to the wild type strain.

The terms “integration” and “integration strains,” as used herein, refer to cell strains in which a recombinant DNA is inserted within a native gene or promoter in such a way as to promote expression or activity of the recombinant DNA. Insertions of recombinant DNA can include proteins or DNA sequences from other species or DNA from the same species inserted into a non-native location to promote transcription and/or translation of a particular gene.

The term “exogenous” indicates that the protein or nucleic acid is a non-native molecule introduced from outside the organism or system, without regard to species of origin. For example, an exogenous protein may be expressed from a recombinant DNA transfected into a cell, a non-native promoter may be introduced to an existing DNA, or a non-native construct may be controlled by a native promoter.

As used herein “recombinant” is relating to, derived from, or containing genetically engineered material. Recombinant DNA can be carried on a vector or integrated into the chromosome of the host cell. Many vectors are known which can be used in a variety of species. Stable chromosomal integration methods are also well documented.

TABLE 3
SEQUENCES

| Description | SEQ |
|---|---|
| VP26 | 1 |
| VP26 primer+ | 2 |
| VP26 primer− | 3 |
| VP28 | 4 |
| VP28 primer+ | 5 |
| VP28 primer− | 6 |
| rfbJ genomic DNA | 7 |
| Synechocystis PCC 6803 rfbJ primer | 8 |
| Synechocystis PCC 6803 rfbJ primer | 9 |
| Synechocystis PCC 6803 psbA II | 10 |
| Synechocystis PCC 6803 psbA II | 11 |
| primer | |
| Synechocystis PCC 6803 psbA II | 12 |
| primer | |
| ScFv-DB coding region | 13 |
| KDEL | 14 |
| GGSSRSS | 15 |
| SGGGGSGGGGGGSSRS | 16 |
| Synechocystis DB3 cassette | |
| Synechocystis ScFv 35 cassette | |
| Synechocystis ScFv 85 cassette | |
| Synechocystis ScFv 86 cassette | |
| Synechocystis ScFv 306 cassette | |
| Chlamydomonas | |
| Synechocystis PCC 6803 | NC_000911 |
| White Spot Syndrome Virus | NC_003225 |

A gene or cDNA may be “optimized” for expression in the photosynthetic microbes Synechocystis, C. reinhardtii or other species using the codon bias for the species. Various nucleotides can encode a single peptide sequence. Understanding the inherent degeneracy of the genetic code allows one of ordinary skill in the art to design multiple nucleotides which encode the same amino acid sequence. NCBI™ provides codon usage databases for optimizing DNA sequences for protein expression in various species.

In calculating “% identity” the unaligned terminal portions of the query sequence are not included in the calculation. The identity is calculated over the entire length of the reference sequence, thus short local alignments with a query sequence are not relevant (e.g., % identity=number of aligned residues in the query sequence/length of reference sequence). Alignments are performed using BLAST homology alignment as described by Tatusova T A & Madden T L (1999) FEMS Microbiol. Lett. 174:247-250. The default parameters were used, except the filters were turned OFF. As of Jan. 1, 2001 the default parameters were as follows: BLASTN or BLASTP as appropriate; Matrix=none for BLASTN, BLOSUM62 for BLASTP; G Cost to open gap default=5 for nucleotides, 11 for proteins; E Cost to extend gap [Integer] default=2 for nucleotides, 1 for proteins; q Penalty for nucleotide mismatch [Integer] default=−3; r reward for nucleotide match [Integer] default=1; e expect value [Real] default=10; W word size [Integer] default=11 for nucleotides, 3 for proteins; y Dropoff (X) for blast extensions in bits (default if zero) default=20 for blastn, 7 for other programs; X dropoff value for gapped alignment (in bits) 30 for blastn, 15 for other programs; Z final X dropoff value for gapped alignment (in bits) 50 for blastn, 25 for other programs. This program is available online at NCBI™ (www.ncbi.nlm.nih.gov/BLAST/).

Common restriction enzymes and restriction sites are found at NEB® (NEW ENGLAND BIOLABS®, www.neb.com) and INVITROGEN® (www.invitrogen.com) as well as other commercial enzyme suppliers. ATCC®, AMERICAN TYPE CULTURE COLLECTION™ (www.atcc.org), DSMZ®, DEUTSCHE SAMMLUNG VON MIKROORGANISMEN UND ZELLKULTUREN™ (www.dsmz.de), KBIF®, KOREAN BIOLOGICAL RESOURCE CENTER™ (kbif.kribb.re.kr), and WDCM®, WORLD DATA CENTRE FOR MICROORGANISMS™ (wdcm.nig.ac.jp) have extensive collections of cell strains that are publicly available. NEB®, INVITROGEN®, ATCC®, DSMZ®, KBIF®, and WDCM® databases are incorporated herein by reference.

“Shuttle vector” is a vector with two or more origins of replication for different species. This allows the vector to replicate in more than one species. Over 200 shuttle vectors are available through ATCC® alone. A shuttle vector can be generated for any species by cloning an origin of replication into a plasmid that already contains an origin for another species.

### EXAMPLE 1

### Materials and Methods

PCR was performed using AMPLITAQ® Gold DNA polymerase kit (APPLIED BIOSYSTEMS™, CA, USA) and a MASTERCYCLER® Gradient thermal cycler (EPPENDORF™, Hamburg, Germany). The PCR reaction was carried out in 100 μl of solution containing 10 μl Roche 10×PCR buffer II, 1 mM MgCl₂, 100 μM dNTP, 0.25 μM each primer, 10 ng genomic DNA as template, and 5 U polymerase. The PCR amplification cycles consisted of denaturation at 94° C. for 45 sec (1 cycle), followed by 29 cycles of 94° C. 45 sec, 53° C. 45 sec and 72° C. 45 sec, the last extension was at 72° C. for 5 min. PCR products are resolved on 1.5% agarose gel stained with ethidium bromide and purified using CONCERT™ PCR purification kit (LIFETECHNOLOGIES™).

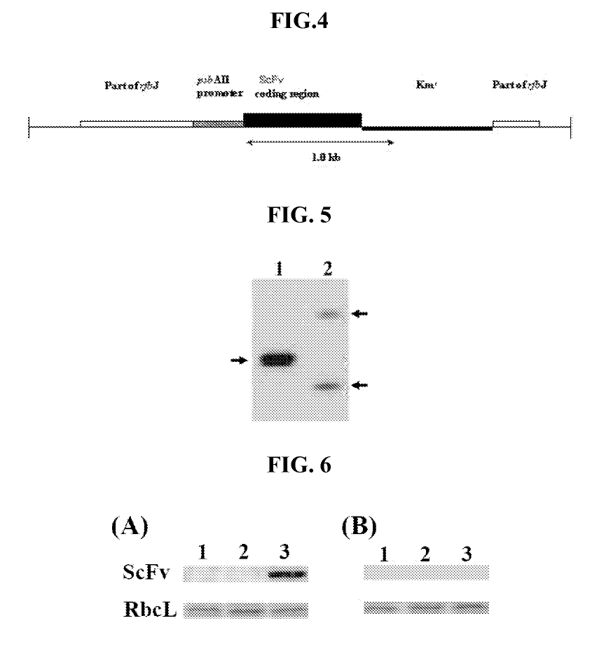

The recombinant DNA cassette illustrated in FIG. 4 was constructed by assembling within the coding region for rjbJ (SEQ ID NO: 7), the promoter region of Synechocystis PCC 6803 psbA II (SEQ ID NO: 10), a kanamycin resistance gene (Km^{R}). The rjbJ coding sequence and flanking DNA (SEQ ID NO: 7) was amplified from Synechocystis PCC 6803 genomic DNA using primer pair SEQ ID NO: 8 and SEQ ID NO: 9. The psbA II promoter (SEQ ID NO. 10), a native promoter that induces constitutive protein expression, was PCR amplified from Synechocystis PCC 6803 using primers SEQ ID NO: 11 and SEQ ID NO: 12.

Kinetics experiments were conducted using BIACORE™ 2000. Ligand-BSA is bound to the chip. Subsequent antibodies that bind to the ligand are captured by the immobilized antigen-BSA conjugate. The flow rate was 30 μl/min. Rate constants were obtained by averaging 3 replicate measurements. The control experiments were done using blank chip for non-specific binding.

Viral DNA was purified according to the procedure reported by Yang et al., (2001). Briefly, WSSV virions were isolated from Procambarus clarkii infected with WSSV. Viral infection is confirmed by SDS-PAGE of virion proteins (FIG. 2A). VP 26 and VP 28 are marked in Lane 2. Lane 1 is the protein size marker with the molecular weight in kDa marked for some bands. Purified rVP26 from the crude lysate of VP26-baculovirus infected Sf21 cells (FIG. 2B) Lane 1 is crude lysate, Lanes 2 and 3 are Ni-NTA wash, Lanes 4,5,6, and 7 are consecutive elutions of VP26 from the loaded column, Marker is in Lane M.

### EXAMPLE 2

### Expression of FcSv in Chlamydomonas

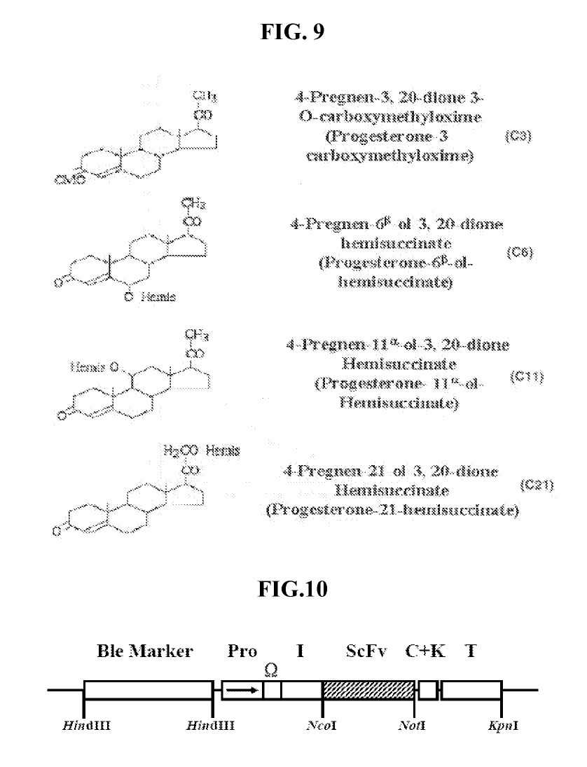

C. reinhardtii 950FcSv was generated by introducing a recombinant DNA cassette (FIG. 10) into the wild type C. reinhardtii. The box filled with crosshatch represents the coding sequence for ScFv with codons optimized for expression in C. reinhardtii, Ble is the bacterial bleomycin resistance gene serving as a dominant selectable marker (Stevens et al. 1996), Pro is the promoter region of RbcS2 in C. reinhardtii(Goldschmidt-Clermont and Rahire. 1986), Ω is the 5′untranslated leader (omega sequence) of tobacco mosaic virus (Schmitz et al. 1996), I is the first intron of RbcS2 (Lumbreras et al. 1998), C+K represent the ER retention sequence KDEL (SEQ ID NO: 14; Napier et al. 1992), T is the terminator of RbcS2 gene of C. reinhardtii. The coding sequence for ScFv was inserted using the NcoI site and NotI site.

In another embodiment, the present invention provides the details for the construction of a recombinant DNA molecule cassette for the generation of Chlamydomonas reinhardtii 950ScFv strain. The positioning of TMV omega sequence, the first intron of RbcS2 and the ER retention sequence KDEL (SEQ ID NO: 14) at shown in FIG. 10 facilitates the quantitative production of functional ScFv.

### EXAMPLE 3

### Expression of ScFv-DB3 in Synechocystis

To investigate the production of ScFvs in cyanobacteria, the coding region for progesterone binding ScFvs DB3 was generated, incorporated into Synechocystis PCC 6803, and expressed in culture. Results indicate that functional DB3 was expressed in Synechocystis 6803 ScFvs DB3 and has a very high specificity for progesterone.

Synthesis of ScFvs DB3 Coding Sequence

An expression cassette for ScFvs DB3 in Synechocystis was designed which incorporated a 3′ flanking region of native genomic DNA, a promoter, an expression construct for the ScFvs DB3, a selection cassette, a termination sequence, and a 5′ flanking region of native genomic DNA. The ScFv-DB coding region (SEQ ID NO: 13) was synthesized based on the published sequence of DB3 (He et al. 1995) using the optimal codon bias for Synechocystis and incorporating an ATG start codon (instead of TGT). The coding region was inserted into the StuI restriction site of the recombinant DNA cassette molecule illustrated in FIG. 4 and the sequence of the final construct was confirmed by DNA sequencing. The recombinant DNA cassette was transformed into Synechocystis PCC6803 to generate Synechocystis 6803 ScFv-DB3. Integration of the DNA cassette was confirmed by serial plating and finally Southern blot analysis was used to confirm insertion of the ScFv construct at the target site as illustrated in FIG. 5. Synechocystis PCC 6803 (lane 1) and Synechocystis ScFv (Lane 2) were compared by Southern Blot hybridization. The genomic DNA for both strains were digested with the restriction enzyme, BstN1, and probed with the full length coding region of rjbJ. Arrow points to each hybridization band.

To confirm production of active ScFv-DB3, the cells were cultured for various times and assessed at different growth stages. Samples were pelleted and resuspended in lysis buffer (20 mM MES/NaOH (pH 6.5), 5 mM MgCl2, 5 mM CaCl2, 20% glycerol (v/v), 1 mM PMSF and 5 mM benzamide). ScFv-DB3 was purified from supernatant by using protein L affinity resin (Pierce) according to the manufacturer's instructions. Translation and accumulation of ScFv-DB3 in Synechocystis 6803 ScFv-DB3 was detected by Western blot using rabbit anti mouse IgG (Fab) detected with goat anti rabbit alkaline phosphatase conjugated IgG as shown in FIG. 6. Antibody binding was detected by using rabbit anti mouse IgG (Fab) that was again detected with goat anti rabbit alkaline phosphatase conjugated IgG. ScFv expression is demonstrated at a variety of harvest times to identify optimal expression and purification conditions. Panel A shows the accumulation of ScFv-DB3 in Synechocystis 6803ScFv-DB3 in the stationary phase culture (Lane 3) but not in the lag phase (Lane 1) and the log phase (lane 2) cultures comparing with the level of the large subunit of ribulose bisphosphate carboxylase (RbcL) under the parallel growth condition. Panel B shows the result obtained from Synechocystis PCC 6803 subjected to parallel treatment, ScFv-DB3 band was not detected, the level of RbcL was not significantly different from that of Synechocystis 6803ScFv-DB3.

Specificity of Synechocystis Produced ScFvs DB3

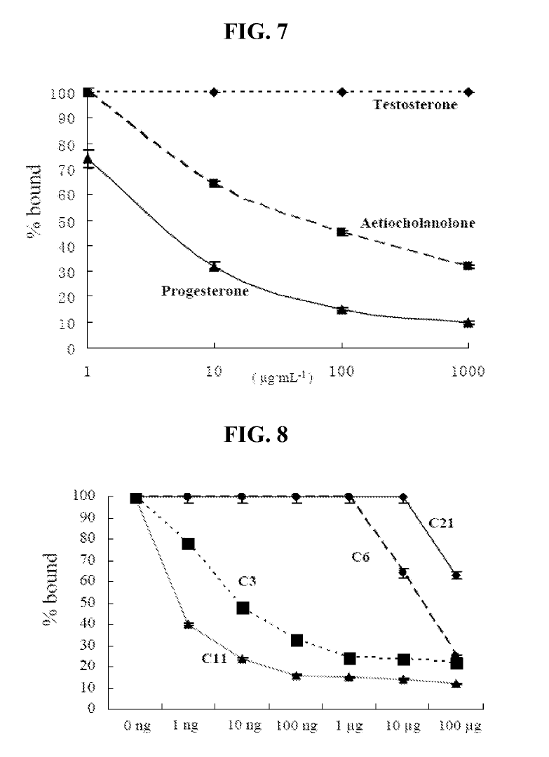

The association constant and specificity of ScFv-DB3 produced by Synechocystis 6803 ScFv-DB3 were measured by using BIACORE™ 2000. Briefly, ligand (e.g. progesterone-BSA) was amino-coupled onto the chip. ScFv-DB3 was subsequently captured by immobilized ligand. The flow rate was 30 μl/min. The rate constants were obtained by averaging 3 replicates. Control experiments were done using blank chip to detect non-specific binding. A comparison of the binding affinities for progesterone, testosterone, and aetiocholanolone demonstrate that ScFv-DB3 produced by Synechocystis 6803 ScFv-DB3 has high affinity to progesterone and low affinity for testosterone and aetiocholanolone (FIG. 7). Further, specificity analysis conducted with progesterone and various progesterone conjugates (FIG. 10) demonstrates that the recombinant ScFv-DB3 produced by Synechocystis 6803 ScFv-DB3 has high specificity for progesterone (FIG. 8). Thus, Synechocystis PCC 6803 was able to produce large quantities of active progesterone binding DB3 single-chain antibodies.

### EXAMPLE 4

### Generation of ScFv VP26

VP26 specific ScFvs proteins are generated by expressing large quantities of recombinant VP26 (r-VP26) purifying the r-VP26 protein, inducing an immune response in mice, generating hybridomas, amplifying DNA encoding light and heavy chain antibodies, linking those DNAs with a flexible linker, and transferring the DNA encoding an ScFv to an expression system. Additionally the present invention provides methods of producing and purifying r-VP26 of WSSV, antibodies directed to r-VP26, and recombinant ScFv that specifically bind VP26.

In another embodiment, the present invention provides the condition of immunizing mice using the r-VP26; the cloning of ScFv fragments for r-VP26; and the subsequent construction of ScFvs and the preparation of ScFv phage display libraries for ScFv r-VP26.

In another embodiment, the present invention provides conditions for the panning and selection of the high affinity ScFvs for r-VP26 of WSSV; methods for determining the in vivo neutralization effect of ScFv r-VP26 for WSSV infection.

In another embodiment, the present invention provides particular growth conditions that facilitated the expression of foreign ScFv in Synechocystis 6803ScFv.

In another embodiment, the present invention provides the method of testing the specificity and affinity of ScFv produced by photosynthetic microbes.

In another embodiment, the present invention provides method of testing the in vivo neutralization effect of ScFv.

Expression of Recombinant VP26

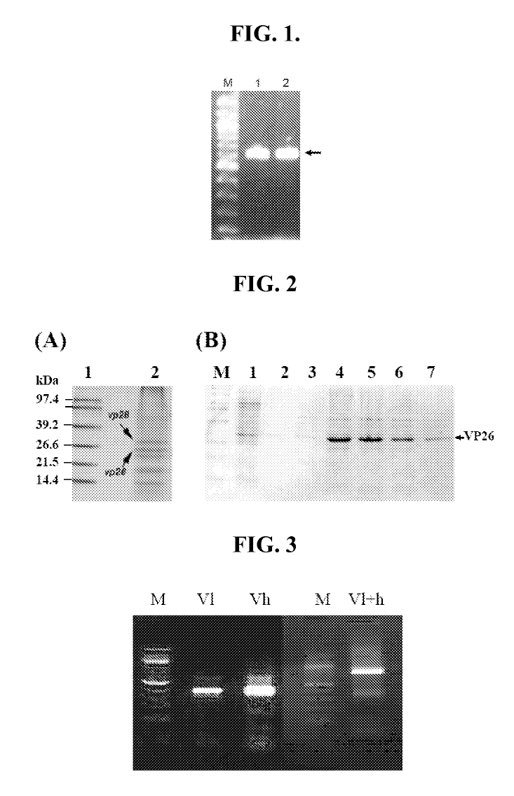

The coding sequence for VP26 (SEQ ID NO: 1) was PCR amplified from WSSV virion DNA using VP26 primers (SEQ ID NO 2 and 3). Amplification of VP26 coding sequence is demonstrated by gel electrophoresis (FIG. 1). Lanes 1 and 2 show PCR amplified VP26 DNA (indicated by the arrow) and 100 bp marker is shown in lane M. The PCR product was cloned into T-vector to generate VP26-TA(11). The inserted VP26 sequence was confirmed by sequencing. The VP26 insert was excised by restriction with NcoI and XbaI, gel-purified using the GIBCOBRL® CONCERT™ Matrix Gel Extraction System, and ligated into PFASTBAC™ HTa vector to generate pFastBacHta-VP26. The pFastBacHta-VP26 was transformed into MAX EFFICIENCY® DH10Bac™ Cells with bacmid bMON14272 and a BAC-TO-BAC™ helper plasmid (INVITROGEN®). The helper plasmid expressing a transposase induces recombination of mini-Tn7 elements in pFastBacHta-VP26 with mini-Tn7 attachment sites in bMON14272. This generates an r-VP26 with His-Tag. The recombinant bacmids containing VP26-His tag were transfected into sf21 cells using CELLFECTIN® reagent to obtain P1 viral stock. The P1 viral stock was amplified in Grace-PS medium and used for protein expression. The expressed r-VP26 was purified by using the Ni-NTA Purification System (INVITROGEN™) and its purity checked by SDS-PAGE.

In another embodiment, the present invention provides a PCR primer set for amplifying region corresponding to SEQ ID NO: 1, a partial genomic DNA sequence of WSSV coding for VP26. The primer set comprising a first primer having a sequence corresponding or complementary to a sequence corresponding to SEQ ID NO: 2; and a second primer having a sequence corresponding or complementary to a sequence corresponding to SEQ ID NO: 3.

Generation of Anti-VP26 Hybridomas

Recombinant VP26 was treated with ACTEV™ protease (INVITROGEN™) at 27° C. overnight to remove the 6× His-Tag at the N-terminus. 8-weeks old female balb-c mice were injected with 75 μg r-VP26 in 600 μl PBS mixed with equal volume of Complete Freund Adjuvant (CFA) (SIGMA®).

Purified rVP26 was used to immunize 8-week old female balb-c mice for the subsequent production of ScFvs for rVP26 using the Mouse ScFv Module, RECOMBINANT PHAGE ANTIBODY SYSTEM™ (RPAS™, AMERSHAM®), according to Barbas III et al. (2001). The teachings of which are incorporated herein by reference.

One month after the primary immunization, each mouse received a booster injection intraperitoneally with 50 μg r-VP26 in 500 μl PBS mixed with equal volume of incomplete CFA. One month was the typical interval between each booster.

The immune serum from each immunized mouse was titered by ELISA using 96-well ELISA plates (Corning) coated with 100 μl PBS containing 0.1 μg r-VP26 at 4° C. overnight. Each well was washed twice with 100 μl PBS, blocked with PBS+5% reconstituted non-fat milk at 37° C. for 1 hour, and rinsed with 100 μl PBS twice.

Fifty μl serially diluted immune serum was added into each well and incubated at 37oC for 1 hr washed with 100 μl PBS 3 times, 50 μl alkaline phosphatase conjugated-goat anti mouse antibody (1:2500) was applied per well and the plate incubated in 37° C. for 1 hour. Each well was washed with 100 μl PBS for 4 times, 50 μl alkaline phosphatase developing solution, freshly prepared by dissolving one 5-mg PNPP tablet and one Tris-buffer tablet in 10 ml double distill water, was applied per well. The plate was incubated at room temperature in darkness for 1-3 hours and OD405 was taken with an ELISA plate reader. Each sample was analyzed in triplicates.

The spleen from a mouse with high titer was dissected out and placed in 5 ml Trizol Reagent (INVITROGEN™) for the isolation of total RNA, mRNA was then isolated from the total RNA using the E.Z.N.A.® mRNA Enrichment Kit (OMEGA BIO-TEK®) according to manufacturer's instruction.

The first strand cDNA was synthesized using the Mouse ScFv Module RPAS™. The heavy chains and light chains variable regions were amplified by using RS primer mix and light chain primer mix Mouse ScFv module (RPAS™) respectively with AMPLITAQ®. The heavy chains and light chains variable regions were re-amplified with VH 5′ sense primers and VH 3′ reverse primers, Vk5′ sense primers and Vk3′ revese primers, Vl5′ sense primers and Vl3′ reverse primers according to Barbas III et al. (2001), using the VENT™ polymerase (NEB®). The teachings of which are incorporated herein by reference. Both the short linker reverse primers and long linker reverse primers were used for the construction of the short linker libraries and long linker libraries for ScFvs r-VP26 or ScFvs r-VP28 respectively.

The amplified variable heavy and light chains were assembled together by overlapping extension PCR using the RPAS™ Mouse ScFv module and the AMPLITAQ® as shown in FIG. 3. Both the short linker reverse primers and long linker reverse primers were used for the construction of the short linker libraries and long linker libraries respectively. The cDNA libraries encompassing the ScFv genes linked with 7 amino acids linker, GGSSRSS (SEQ ID NO: 15), or 16 amino acids linker, SGGGGSGGGGGGSSRS (SEQ ID NO: 16), were prepared. These libraries were displayed on phages using RPAS™ according to Barbas III, et al. (2001).

After repeated panning using Nunc MaxiSorp™ flat-bottom 96 well plates coated with purified VP26, several high affinity ScFvs for VP26 proteins were identified. Using BIACORE™, ScFvs binding different specific epitopes were isolated. High affinity ScFvs strong enough for early detection were identified and isolated.

### EXAMPLE 5

### Generation of ScFv VP28

The Coding region for VP28 (SEQ ID NO: 4) was amplified by PCR using the primer pair of SEQ ID NO: 5/SEQ ID NO: 6 from the WSSV DNA purified from a China isolate of WSSV.

In another embodiment, the present invention provides a PCR primer set for amplifying region corresponding to SEQ ID NO: 4, a partial genomic DNA sequence of WSSV coding for VP28. The primer set comprising a first primer having a sequence corresponding or complementary to a sequence corresponding to SEQ ID NO: 5; and a second primer having a sequence corresponding or complementary to a sequence corresponding to SEQ ID NO: 6.

The coding region for 6×His-tag was added at the N-terminus to the coding region of VP28 to generate r His-VP26 and r His-VP28 respectively.

The r His-VP28 recombinant DNA was introduced into the insect cell lines sf21 using the BAC-TO-BAC Baculovirus Expression System (INVITROGEN™) for the production of r His-r His-VP28 respectively.

The recombinant His-VP28 were purified by using the Ni-NTA Purification System (INVITROGEN™) and treated with AcTEV protease (INVITROGEN™) at 27° C. overnight to remove the His Tag to generate rVP28.

Purified rVP28 was used to immunize 8-week old female balb-c mice for the subsequent production of ScFvs for rVP28 using the Mouse ScFv Module, RECOMBINANT PHAGE ANTIBODY SYSTEM™ (RPAS™, AMERSHAM®), according to Barbas III et al. (2001). The teachings of which are incorporated herein by reference.

One month after the primary immunization, each mouse received a booster injection intraperitoneally with 50 μg r-VP28 in 500 μl PBS mixed with equal volume of incomplete CFA. One month was the typical interval between each booster.

Generation of Anti-VP28 Hybridomas

Recombinant VP28 was treated with ACTEV™ protease (INVITROGEN™) at 27° C. overnight to remove the 6×His-Tag at the N-terminus. 8-weeks old female balb-c mice were injected with 75 μg r-VP28 in 600 μl PBS mixed with equal volume of Complete Freund Adjuvant (CFA) (SIGMA®).

Purified rVP28 was used to immunize 8-week old female balb-c mice for the subsequent production of ScFvs for rVP28 using the Mouse ScFv Module, RECOMBINANT PHAGE ANTIBODY SYSTEM™ (RPAS™, AMERSHAM®), according to Barbas III et al. (2001). The teachings of which are incorporated herein by reference.

One month after the primary immunization, each mouse received a booster injection intraperitoneally with 50 μg r-VP28 in 500 μl PBS mixed with equal volume of incomplete CFA. One month was the typical interval between each booster.

The immune serum from each immunized mouse was titered by ELISA using 96-well ELISA plates (Corning) coated with 100 μl PBS containing 0.1 μg r-VP28 at 4° C. overnight. Each well was washed twice with 100 μl PBS, blocked with PBS+5% reconstituted non-fat milk at 37° C. for 1 hour, and rinsed with 100 μl PBS twice.

Fifty μl serially diluted immune serum was added into each well and incubated at 37oC for 1 hr washed with 100 μl PBS 3 times, 50 μl alkaline phosphatase conjugated-goat anti mouse antibody (1:2500) was applied per well and the plate incubated in 37° C. for 1 hour. Each well was washed with 100 μl PBS for 4 times, 50 μl alkaline phosphatase developing solution, freshly prepared by dissolving one 5-mg PNPP tablet and one Tris-buffer tablet in 10 ml double distill water, was applied per well. The plate was incubated at room temperature in darkness for 1-3 hours and OD405 was taken with an ELISA plate reader. Each sample was analyzed in triplicates.

The spleen from a mouse with high titer was dissected out and placed in 5 ml Trizol Reagent (INVITROGEN™) for the isolation of total RNA, mRNA was then isolated from the total RNA using the E.Z.N.A.® mRNA Enrichment Kit (OMEGA BIO-TEK®) according to manufacturer's instruction.

The first strand cDNA was synthesized using the Mouse ScFv Module RPAS™. The heavy chains and light chains variable regions were amplified by using RS primer mix and light chain primer mix Mouse ScFv module (RPAS™) respectively with AMPLITAQ®. The heavy chains and light chains variable regions were re-amplified with VH 5′ sense primers and VH 3′ reverse primers, Vk5′ sense primers and Vk3′ reverse primers, Vl5′ sense primers and Vl3′ reverse primers according to Barbas III et al. (2001), using the VENT™ polymerase (NEB®). The teachings of which are incorporated herein by reference. Both the short linker reverse primers and long linker reverse primers were used for the construction of the short linker libraries and long linker libraries for ScFvs r-VP28 respectively.

The amplified variable heavy and light chains were assembled together by overlapping extension PCR using the RPAS™ Mouse ScFv module and the AMPLITAQ® as shown in FIG. 3. Both the short linker reverse primers and long linker reverse primers were used for the construction of the short linker libraries and long linker libraries respectively. The cDNA libraries encompassing the ScFv genes linked with 7 amino acids linker, GGSSRSS (SEQ ID NO: 15), or 16 amino acids linker, SGGGGSGGGGGGSSRS (SEQ ID NO: 16), were prepared. These libraries were displayed on phages using RPAS™ according to Barbas III, et al. (2001).

After repeated panning using NUNC MAXISORP™ flat-bottom 96 well plates coated with purified VP28, several high affinity ScFvs for the respective VP proteins were identified. Using BIACORE™, ScFvs binding different specific epitopes were isolated. High affinity ScFvs strong enough for early detection were identified and isolated.

### EXAMPLE 6

### Purification of ScFv VP26 and ScFv VP28

Another aspect of the invention provides for the low cost production of functional ScFvs in photosynthetic microbes including prokaryotic blue-green alga, i.e. Synechocystis, and the eukaryotic green alga, i.e. C. reinhardtii, via expression of integrated recombinant DNA coding for ScFv.

Heterotrophic algae provide an excellent background for the inexpensive production of large quantities of ScFvs. The heterotrophic algae, Synechocystis 6803ScFv or C. reinhardtii 950FcSv, were grown to density. The cells were pelleted and resuspended in lysis buffer containing 20 mM MES/NaOH (pH 6.5), 5 mM MgCl₂, 5 mM CaCl₂, 20% glycerol (v/v), 1 mM PMSF and 5 mM benzamide. Cellular debris was removed by centrifugation. ScFv was purified from supernatant using the protein L affinity resin (PIERCE™) according to the manufacturer's protocol. Purified ScFv can be lyophilized for long term storage, stored in solution and frozen, or maintained in solution at 4° C. for use.

Purity and specificity of the final ScFv solutions is measured after resuspension in aqueous buffer. Purity is assessed by SDS-PAGE stained with either silver stain or other standard protein stain (e.g. coomassie blue). A variety of commercial kits are available for SDS-PAGE, protein staining, and quantitation of protein in the sample. Specificity of the ScFv is measured using the BIACORE™ 2000 protocol previously described.

### EXAMPLE 7

### Neutralization of WSSV Infection

In another embodiment, the present invention provides a collection of ScFvs that showed high affinity to r-VP28 or r-VP26 of WSSV and demonstrated significant in vivo neutralization effect either alone or in different combinations for WSSV infection. An in vivo neutralization assay using the crayfish Procambarus clarkii was developed to detect the effect of each ScFv and the effect of combining different ScFvs on WSSV infection.

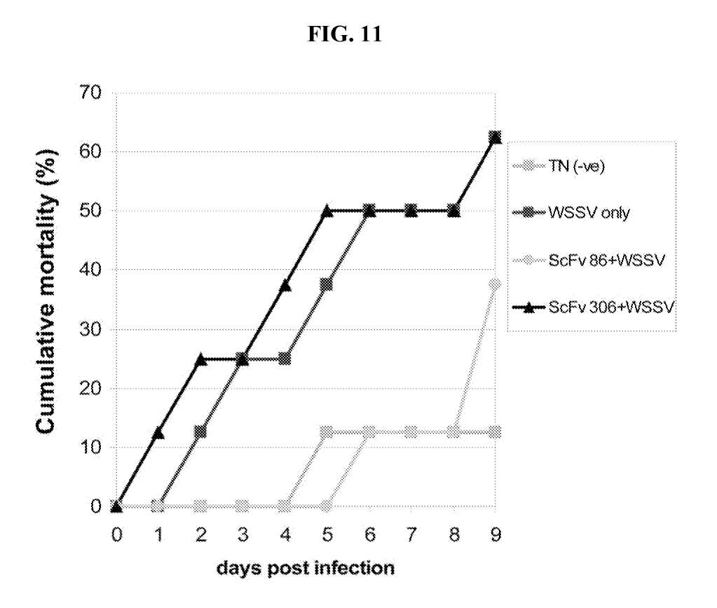

Healthy Procambarus clarkii were acclimatized to 22° C. and 14 hr: 10 hr light:dark cycle, and fed WELSI GRAN™ granulated catfish feed (TROPICAL®). The minimal acclimation period was three days. At least eight crayfish were included with each group and each test was performed in duplicate. For the treatment group, fifty μl TN buffer solution containing 5×10⁷ WSSV virions and 2 μg purified ScFv was injection into each crayfish between the third and fourth abdominal segments using a 30G needle. For the control group, each crayfish received 50 μl TN buffer containing 5×10⁷ WSSV virions and 2 μg purified non-binding ScFv. Cumulative mortality was recorded daily. An example of the result is shown in FIG. 11.

In vivo neutralization is observed using the ScFv 85 as shown in FIG. 1. ScFv 86 delayed the onset of mortality and reduced the cumulative mortality of WSSV infections. To ensure infectivity of the WSSV virions, a mixture of WSSV and ScFv 306, that has no affinity for WSSV, were injected into the shrimp. Another group of shrimp were not exposed to WSSV.

ScFv 35 and ScFv 86 were shown to delay the onset of WSSV infection and reduced the accumulated mortality. These ScFvs provide prophylactic reagents against WSSV infection.

### REFERENCES

All references are listed herein for the convenience of the reader. Each is incorporated by reference in its entirety.
1. U.S. Pat. No. 5,863,765, Berry, et al., “Production in yeasts of stable antibody fragments” (1999).
2. Guzmán & Valle, “Infectious disease in shrimp species with aquaculture potential,” Recent Res. Dev. Microbiol. 4:333-48 (2000).
3. Barbas, et al., Phage Display: A Laboratory Manual. Cold Spring Harbor Laboratory Press, Cold Spring Harbor, N.Y. (2001).
4. Boder, et al., “Directed evolution of antibody fragments with monovalent femtomolar antigen-binding affinity,” Proc. Natl. Acad. Sci., USA 97:10701-5 (2000).
5. Chen, “Current status of shrimp aquaculture in Taiwan.” In Browdy Cl, Hopkins J S (eds) Swimming through troubled water. Proceedings of the special session in shrimp farming. Aquaculture '95. World Aquaculture Society, Baton Rouge La. USA, pp. 29-34 (1995).
6. Chou, et al., “Pathogenicity of a baculovirus infection causing white spot syndrome in cultured penaeid shrimp in Taiwan,” Dis. Aquat. Organ. 23:165-73 (1995).
7. Goldschmidt-Clermont & Rahire, “Sequence, evolution and differential expression of the two genes encoding variant small subunits of ribulose bisphosphate carboxylase/oxygenase in Chlamydomonas reinhardtii,” J. Mol. Biology. 191:421-32 (1986).
8. He, et al., “Characterization of a progesterone-binding, three-domain antibody fragment (Vh/K) expressed in E. coli,” Immunol. 84:662-8 (1995).
9. Huang, et al., “Baculoviral hypodermal and hematopoietic necrosis, study on the pathogen and pathology of the shrimp explosive epidemic disease of shrimp,” Mar. Fish. Res. 16:1-10 (1995).
10. Inouye, et al., “The penaeid rod-shaped virus (PRDV) which caused penaeid acute viremia (PAV),” Fish Pathology 31:39-45 (1996).
11. Lightner “A Handbook of shrimp pathology and diagnostic procedure for diseases of cultured peaneid shrimp,” The World Aquaculture Society, Baton Rouge, La., USA. 304 p. (1996).
12. Lo, et al., “Specific genomic fragment analysis of different geographical clinical samples of shrimp white spot syndrome virus,” Dis. Aquat. Organ. 35:175-85 (1999).
13. Lumbreras, et al., “Efficient foreign gene expression in Chlamydomonas reinhardtii mediated by an endogenous intron,” Plant J. 14:441-7 (1998).
14. Napier, et al., “Immunological evidence that plants use both HDEL and KDEL for targeting proteins to the endoplasmic reticulum,” J. Cell Sci. 102:261-71 (1992).
15. Schmitz, et al., “Non-canonical translation mechanisms in plants: efficient in vitro and in planta initiation at AUU codons of the tobacco mosaic virus enhancer sequence,” Nucl. Acids Res. 24:257-63 (1996).
16. Stevens, et al., “The bacterial phleomycin resistance gene ble as a dominant selectable marker in Chlamydomonas,” Mol. Gen. Genet. 251:23-30 (1996).
17. Wongteerasupaya, et al., “A non-occluded, systemic baculovirus that occurs in cells of ectodermal and mesodermal origin and causes high mortality in the black tiger prawn Penaeus monodon,” Dis. Aquat. Organ. 21:69-77 (1995).
18. Yang, et al., “Complete genome sequence of the shrimp white spot bacilliform virus,” J. Vir. 75:11811-20 (2001).

1. A heterotrophic algae comprising a recombinant DNA encoding a single chain fragment variable (ScFv) antibody operably linked to a promoter, wherein said heterotrophic algae produces an active ScFv antibody.

2. The heterotrophic algae of claim 1, wherein said heterotrophic algae is selected from the group consisting of Chlamydomonas, Synechococcus, Thermosynechococcus, Synechocystis, Euglena, Gloeobacter, Nostoc, Anabaena, Trichodesmium, Prochlorococcus, Chlorella, and Pseudendoclonium.

3. The heterotrophic algae of claim 1, wherein said ScFv antibody specifically binds to progesterone, VP26, or VP28, White Spot Syndrome Virus (WSSV), white spot baculovirus (WSBV), cholera, systemic ectodermal and mesodermal baculovirus (SEMBV), penaeid rod-shaped DNA virus (PRDV), hypodermal and hematopoietic necrosis baculovirus (HHNBV), or other viruses.

4. The heterotrophic algae of claim 1, wherein said recombinant DNA is a site-specific integration cassette comprising (A) a promoter, (B) an ScFv antibody coding region, and (C) a stop codon, wherein said recombinant DNA is integrated in the host cell.

5. The heterotrophic algae of claim 1, wherein said ScFv antibody is selected from the group consisting of ScFv 35, ScFv 85, ScFv 86, DB3, or other ScFv antibody optimized for expression in said heterotrophic algae.

6. The heterotrophic algae of claim 1, wherein said algae is Synechocystis, wherein said recombinant DNA is a site-specific integration cassette comprising a Synechocystis psbA II promoter, an ScFv antibody coding region, and a psbA II stop codon wherein said integration cassette allows specific chromosomal integration of the recombinant DNA.

7. The heterotrophic algae of claim 1, wherein said algae is Chlamydomonas, wherein said recombinant DNA is a site-specific integration cassette comprising, a promoter region of RbcS2 promoter, the 5′untranslated leader of tobacco mosaic virus, the first intron of RbcS2, an ScFv antibody coding region and the ER retention sequence KDEL (SEQ ID NO: 14), wherein said integration cassette allows specific chromosomal integration of the recombinant DNA.

8. A method of inhibiting an aquaculture pathogen comprising:
a. generating a heterotrophic algae comprising a recombinant DNA encoding a single chain fragment variable (ScFv) antibody operably linked to a promoter, wherein said heterotrophic algae produces an active ScFv antibody,
b. inoculating an aquaculture with the heterotrophic algae (a), and
c. expressing said ScFv antibody thereby inhibiting said aquaculture pathogen.

9. The method of claim 8, wherein said heterotrophic algae is selected from the group consisting of Chlamydomonas, Synechococcus, Thermosynechococcus, Synechocystis, Euglena, Gloeobacter, Nostoc, Anabaena, Trichodesmium, Prochlorococcus, Chlorella, and Pseudendoclonium.

10. The method of claim 8, wherein said ScFv antibody specifically binds to progesterone, VP26, or VP28, White Spot Syndrome Virus (WSSV), white spot baculovirus (WSBV), cholera, systemic ectodermal and mesodermal baculovirus (SEMBV), penaeid rod-shaped DNA virus (PRDV), hypodermal and hematopoietic necrosis baculovirus (HHNBV), or other viruses.

11. The method of claim 8, wherein said recombinant DNA is a site-specific integration cassette comprising (A) a promoter from said heterotrophic algae, (B) an ScFv antibody coding region, (C) a stop codon from said heterotrophic algae, and (D) about 400 nucleotides of host DNA.

12. The method of claim 8, wherein said ScFv antibody is selected from the group consisting of ScFv 35, ScFv 85, ScFv 86, DB3, or other ScFv antibody optimized for expression in said heterotrophic algae.

13. The method of claim 8, wherein said algae is Synechocystis, wherein said recombinant DNA is a site-specific integration cassette comprising a Synechocystis psbA II promoter, an ScFv antibody coding region, and a psbA II stop codon wherein said integration cassette allows specific chromosomal integration of the recombinant DNA.

14. The method of claim 8, wherein said algae is Chlamydomonas, wherein said recombinant DNA is a site-specific integration cassette comprising, a promoter region of RbcS2 promoter, the 5′untranslated leader of tobacco mosaic virus, the first intron of RbcS2, an ScFv antibody coding region and the ER retention sequence KDEL (SEQ ID NO: 14), wherein said integration cassette allows specific chromosomal integration of the recombinant DNA.

15. A recombinant single chain fragment variable (ScFv) antibody that binds specifically to an aquaculture pathogen, wherein said ScFv is produced by:
a. generating a heterotrophic algae comprising a recombinant DNA encoding a single chain fragment variable (ScFv) antibody operably linked to a promoter, wherein said heterotrophic algae produces an active ScFv antibody,
b. inoculating an aquaculture with the heterotrophic algae (a), and
c. expressing said ScFv antibody thereby inhibiting said aquaculture pathogen.

16. The ScFv antibody of claim 15, wherein said heterotrophic algae is selected from the group consisting of Chlamydomonas, Synechococcus, Thermosynechococcus, Synechocystis, Euglena, Gloeobacter, Nostoc, Anabaena, Trichodesmium, Prochlorococcus, Chlorella, and Pseudendoclonium.

17. The ScFv antibody of claim 15, wherein said ScFv antibody specifically binds to progesterone, VP26, or VP28, White Spot Syndrome Virus (WSSV), white spot baculovirus (WSBV), cholera, systemic ectodermal and mesodermal baculovirus (SEMBV), penaeid rod-shaped DNA virus (PRDV), hypodermal and hematopoietic necrosis baculovirus (HHNBV), or other viruses.

18. The ScFv antibody of claim 15, wherein said recombinant DNA is a site-specific integration cassette comprising (A) a promoter from said heterotrophic algae, (B) an ScFv antibody coding region, (C) a stop codon from said heterotrophic algae, and (D) about 400 nucleotides of host DNA.

19. The ScFv antibody of claim 15, wherein said ScFv antibody is selected from the group consisting of ScFv 35, ScFv 85, ScFv 86, DB3, or other ScFv antibody optimized for expression in said heterotrophic algae.

20. The ScFv antibody of claim 15, wherein said algae is Synechocystis, wherein said recombinant DNA is a site-specific integration cassette comprising a Synechocystis psbA II promoter, an ScFv antibody coding region, and a psbA II stop codon wherein said integration cassette allows specific chromosomal integration of the recombinant DNA.

21. The ScFv antibody of claim 15, wherein said algae is Chlamydomonas, wherein said recombinant DNA is a site-specific integration cassette comprising, a promoter region of RbcS2 promoter, the 5′untranslated leader of tobacco mosaic virus, the first intron of RbcS2, an ScFv antibody coding region and the ER retention sequence KDEL (SEQ ID NO: 14), wherein said integration cassette allows specific chromosomal integration of the recombinant DNA.


SEQUENCE LISTING (22 sequences)

SEQ ID NO: 1 - DNA, 615 bp, white spot syndrome baculovirus
atggaatttggcaacctaacaaacctggacgttgcaattattgcaatcttgtccattgcaatcattgctctaatcgttatcatggttataatgattgtattcaacacacgtgttggaagaagcgtcgtcgctaattatgatcagatgatgcgagtcccaattcaaagaagggcaaaggtaatgtcaattcgtggagagaggtcctacaatactcctcttggaaaggtggccatgaagaatggtctctccgataaggacatgaaggatgtttctgctgatcttgtcatctctaccgtcacagccccaaggactgatcccgctggcactggggccgagaactctaacatgactttgaagatcctcaacaacactggcgtcgatctcttgatcaacgacattactgttcggccaactgttattgcaggaaacattaagggaaatactatgtcgaacacttacttctcgagcaaggacattaaatcttcatcttcaaaaattaccctcattgacgtgtgcagcaaatttgaagacggcgcagccttcgaagctacaatgaacattggattcacctccaagaatgtgatcgatatcaaggacgaaatcaagaagaagtaa

SEQ ID NO: 2 - DNA, 31 bp, Artificial
  Synthetic primer
catgccatggaatttggcaacctaacaaacc

SEQ ID NO: 3 - DNA, 33 bp, Artificial
  Synthetic primer
gctctagattacttcttcttgatttcgtccttg

SEQ ID NO: 4 - DNA, 615 bp, white spot syndrome baculovirus
atggatctttctttcactctttcggtcgtgtcggccatcctcgccatcactgctgtgattgctgtatttattgtgatttttaggtatcacaacactgtgaccaagaccatcgaaacccacacagacaatatcgagacaaacatggatgaaaacctccgcattcctgtgactgctgaggttggatcaggctacttcaagatgactgatgtgtcctttgacagcgacaccttgggcaaaatcaagatccgcaatggaaagtctgatgcacagatgaaggaagaagatgcggatcttgtcatcactcccgtggagggccgagcactcgaagtgactgtggggcagaatctcacctttgagggaacattcaaggtgtggaacaacacatcaagaaagatcaacatcactggtatgcagatggtgccaaagattaacccatcaaaggcctttgtcggtagctccaacacctcctccttcacccccgtctctattgatgaggatgaagttggcacctttgtgtgtggtaccacctttggcgcaccaattgcagctaccgccggtggaaatcttttcgacatgtacgtgcacgtcacctactctggcactgagaccgagtaa

SEQ ID NO: 5 - DNA, 30 bp, Artificial
  Synthetic primer
catgccatggatctttctttcactctttcg

SEQ ID NO: 6 - DNA, 28 bp, Artificial
  Synthetic primer
gctctagattactcggtctcagtgccag

SEQ ID NO: 7 - DNA, 2450 bp, Synechocystis PCC6803
acctccattgaggtctatcaagcctacgtggactataacgagatgatggccttaaccgaagccctcgtcaccaccgccgcccaagcagtattaggcacgctaaaaattacctaccaaggggaagaaatcgacctcacccctccctggaaacgcatcaccatgcacgaagcggtgcagttagaaacagggatagattttagccaatttacagatctggaaacggccaaacaagcggccacaaaagcgggcattggtgtgccggaagattgtccttcccttgggcatttgttaaaccatgcttttgagcaaaaagtggaagggacgttgatgcaacccacttttatcatcgatttcccggtggaaatttctcccctcgctaagccccatcgcagtaaaccggggctggtggaacggtttgagctatttgtttacggtcgggaattggccaatagtttttccgagctgacagaccccattgatcaacggagtcggttggaagcccaggcggctaaaaaagcggcaggggatttggaagcccacagcgttgatgaagatttcctcacagcgttggaatacggcatgccccctactggtggcttgggcataggcatcgatcgcctggtgatgttgctgaccgattcccccagtattcgagatgtaattgcttttccgttgctgaaaaatcaggaggctggcagcgatggttgatattttcctaggagctaactgtcgagggaatttaattggctcctagacctccttaatttcgaccctgggaattctgagataagccatgcgcttaagctcccgtcttccccgggcaacggaatctaaaattaggccgcaggtgaaaaagacaaagcccaaggacattaaagaagcggcaatgatgactgtgggaaatctcggtactagacccgtttccagataggtaaataccaccggcaacattaaggcgatcgccgacccggaaaagagggcaaagaacacaccaaaaaagaggaatgggcgggtttccttcatcagtaaaaaggaggtacccaaaacccgaaagccatcgctaatggtgtttaacttactttcggagccagaagggcgagattggtataacactggttcttcggcaaagggcagacgtagttctagggtgtggatagttaattccgtttcaatttcaaatccgttggaaatagctgggaagcatttaacaaaacggcgggaaaaaattctgtagcccgacaacatatcgtccaaccgagccccaaattgtaattccacaatgccctttaatagttggttaccactgcggtggccaaaacggtaggcctccttgtccttaacctccgatcgccgagcccccaccaccatatccaattgctcattaatcaggcgatcaatcaatctacccaccgcttcggcttcataggtattgtccccatccaccaaaatgtaaatatccgcttcaatatcagcaaacatgcggcggatcacattgcctttacccggttgtttctcgtaacgaacaatggcccctgcttgctgggcgacggtggcggtgtcatcggtggaacaattgtcgtaaacgtagatggcggcattgggcaactgctggcgaaaatcagtcacaacctgggtgatggtcaaagcttcattacggcaaggaactagcaccgccaaacgataggcactggaaaaagaggttgacaccgttaatttagccaaaaattcggtaaaaataagctgagaaagggtcagagcgcctgtatttttcctaaacgttcataattcaggattttaatagaggcctgtgccaatggttctcagaactggggctaattttatctggccggttgtccctctgcaagtttgtccagccaaagccacgatgataattaattggttaatagggatcacacaggactagcaaacagagtatggccatcgctgttgcaacggtaacccaatccctctaacaacggttggactcccaacaatgccgattgtttggttttcacatcagcgtggatgtcaatgcaaagataggggcgacaggtttttaatgtttccatggccccctggagagcatacagttcaaaaccttcaatatcaattttgatcgctttggggctgagattgttttccccggcaaatttatcaatggtggtgatttccactgcgtaggtagcataattatctaggctaggctccacatctttcgccaaggtagaagtgtccgccgcccaggaaaaacggttatcagtactatattcaatgaattccgcttcccccgtttcatcggcgatcgccaggggaagaactgtaatattttccaggtggttgcccttaacgatttgttcaaggctttgacgggtttggcgagcgggctcaaaggcatagatatggccgttattaccaacaatttctgccaagggaatactaatcaccccgta

SEQ ID NO: 8 - DNA, 36 bp, Artificial
  Synthetic primer
gagagagagagactcgagtacggggtgattagtatt

SEQ ID NO: 9 - DNA, 35 bp, Artificial
  Synthetic primer
gagagagagagaccgcggacctccattgaggtcta

SEQ ID NO: 10 - DNA, 496 bp, Synechocystis PCC6803
agcgttccagtggatatttgctgggggttaatgaaacattgtggcggaacccagggacaatgtgaccaaaaaattcagggatatcaataagtattaggtatatggatcataattgtatgcccgactattgcttaaactgactgaccactgaccttaagagtaatggcgtgcaaggcccagtgatcaatttcattatttttcattatttcatctccattgtccctgaaaatcagttgtgtcgcccctctacacagcccagaactatggtaaaggcgcacgaaaaaccgccaggtaaactcttctcaacccccaaaacgccctctgtttacccatggaaaaaacgacaattacaagaaagtaaaacttatgtcatctataagcttcgtgtatattaacttcctgttacaaagctttacaaaactctcattaatcctttagactaagtttagtcagttccaatctgaacatcgacaaatacataaggaattataaccaa

SEQ ID NO: 11 - DNA, 38 bp, Artificial
  Synthetic primer
gagagagagactcgagttggttataattccttatgtat

SEQ ID NO: 12 - DNA, 33 bp, Artificial
  Synthetic primer
gagagagagaggtaccagcgttccagtggatat

SEQ ID NO: 13 - DNA, 1 bp, Artificial
  Synthetic Single chain Fragment variable antibody
a

SEQ ID NO: 14 - protein, 4 aa, Artificial
  Synthetic ER retention sequence
KDEL

SEQ ID NO: 15 - protein, 7 aa, Artificial
  Synthetic 7 amino acid linker sequence
GGSSRSS

SEQ ID NO: 16 - protein, 16 aa, Artificial
  Synthetic 16 amino acid linker
SGGGGSGGGGGGSSRS

SEQ ID NO: 17 - DNA, 1 bp, Artificial
  Synthetic DB3 single chain fragment variable antibodye expression cassette
a

SEQ ID NO: 18 - DNA, 1 bp, Artificial
  Synthetic ScFv35 single chain fragment variable antibody expression cassette
a

SEQ ID NO: 19 - DNA, 1 bp, Artificial
  Synthetic ScFv 85 single chain fragment variable antibody expression cassette
a

SEQ ID NO: 20 - DNA, 1 bp, Artificial
  Synthetic ScFv 86 single chain fragment variable antibody expression cassette
a

SEQ ID NO: 21 - DNA, 1 bp, Artificial
  Synthetic ScFv 306 single chain fragment variable antibody expression cassette
a

SEQ ID NO: 22 - DNA, 1 bp, Artificial
  Synthetic ScFv 950 single chain fragment variable antibody expression cassette
a